(19) United States
(12) Patent Application Publication
(10) Pub. No.: US 2021/0340278 A1
(43) Pub. Date: Nov. 4, 2021

(54) METHODS AND PHARMACEUTICAL COMPOSITION FOR THE TREATMENT OF MUCOSAL INFLAMMATORY DISEASES

(71) Applicants: INSERM (Institut National de la Santé et de la Recherche Médicale), Paris (FR); Université de Rennes, Rennes (FR); Université de Paris, Paris (FR); Enios Applications Private Limited Company, Kallithea Athens (GR)

(72) Inventors: Eric CHEVET, Rennes (FR); Eric OGIER-DENIS, Paris (FR); Aristotelis CHATZIIOANNOU, Kallithea Athens (GR)

(21) Appl. No.: 17/282,580

(22) PCT Filed: Oct. 3, 2019
(86) PCT No.: PCT/EP2019/076804

§ 371 Date: Apr. 2, 2021

(30) Foreign Application Priority Data
Oct. 4, 2018 (EP) 18306305.6
Apr. 25, 2019 (EP) 19305531.6

(51) Int. Cl. C07K 16/40; A61P 1/00

(52) U.S. Cl. CPC C07K 16/40; A61P 1/00; A61K 2039/505; C07K 2317/76; C07K 2317/565; C07K 2317/34

(57) ABSTRACT

The mucosa is an integrated network of tissues, cells and effector molecules that protect the host from environmental insults and infections. Dysregulation of immunity at mucosal surfaces is thought to lead to mucosal inflammatory diseases such as those affecting the gastrointestinal system (Crohn's disease, ulcerative colitis and irritable bowel syndrome) and respiratory system (asthma, allergy and chronic obstructive pulmonary disorder). Anterior Gradient 2 (AGR2) is a dimeric Protein Disulfide Isomerase (PDI) family member involved in the regulation of protein quality control in the Endoplasmic Reticulum (ER). Its deletion in the mouse intestine increases tissue inflammation and promotes the development of inflammatory bowel disease (IBD). Now the inventors demonstrate that modulation of AGR2 dimer formation yields pro-inflammatory phenotypes notably though the secretion of AGR2 (eAGR2) that promotes monocyte attraction. The inventors show that in IBD and specifically in Crohn's disease, the levels of AGR2 dimerization modulators are selectively deregulated, and this correlates with severity of disease. The inventors thus demonstrate that AGR2 represent systemic alarm signals for pro-inflammatory responses in mucosa. Accordingly, the present invention relates to a method of treating a mucosal inflammatory disease in a subject in need thereof comprising administering to the subject a therapeutically effective amount of an agent which neutralizes the pro-inflammatory activity of eAGR2.

### FIELD OF THE INVENTION

The present invention relates to methods and pharmaceutical composition for the treatment of mucosal inflammatory diseases.

### BACKGROUND OF THE INVENTION

The mucosa (including airway, intestinal, oral and cervical epithelium) is an integrated network of tissues, cells and effector molecules that protect the host from environmental insults and infections. Dysregulation of immunity at mucosal surfaces is thought to be responsible for the alarming global increase in mucosal inflammatory diseases such as those affecting the gastrointestinal system (Crohn's disease, ulcerative colitis and irritable bowel syndrome) and respiratory system (asthma, allergy and chronic obstructive pulmonary disorder). Accordingly, there is a need for novel therapies for the treatment of mucosal inflammatory diseases.

The regulation of protein homeostasis (proteostasis) in the Endoplasmic Reticulum (ER) has recently emerged as an important pathophysiological mechanism involved in the development of different diseases¹. The capacity of the ER to cope with the protein misfolding burden is controlled by the kinetics and thermodynamics of folding and misfolding (also called proteostasis boundary), which are themselves linked to the ER protein homeostasis network capacity². The ER ensures proper folding of newly synthesized proteins through the coordinated action of ER-resident molecular chaperones, folding catalysts, quality control and degradation mechanisms. Anterior gradient 2 (AGR2), a folding catalyst, binds to nascent protein chains, and it is required for the maintenance of ER homeostasis^{3, 4, 5} Loss of AGR2 has been associated with intestinal inflammation^{6, 7}, and several studies have demonstrated that unresolved ER stress leads to spontaneous intestinal inflammation⁸. In mammals, AGR2 is generally expressed in mucus secreting epithelial cells and is highly expressed in Paneth and goblet intestinal progenitor cells, with the highest levels in the ileum and colon^{9, 10, 11}. In goblet cells, AGR2 forms mixed disulfide bonds with Mucin 2 (MUC2), allowing for its correct folding and secretion^{6, 7}. MUC2 is an essential component of the gastrointestinal mucus covering the epithelial surface gastrointestinal tract to confer the first line of defense against commensal bacteria. Knockout of AGR2 inhibits MUC2 secretion by intestinal cells thereby decreasing the amount of intestinal mucus leading to a spontaneous granulomatous ileocolitis, closely resembling human inflammatory bowel disease (IBD)⁷. Accordingly, lowered expression of AGR2 expression and some of its variants were identified as risk factors in IBD¹². However, despite the strong link between AGR2 and the etiology of IBD, the molecular mechanism by which AGR2 regulates its activity and contribute to the development of IBD still remains elusive.

### SUMMARY OF THE INVENTION

The present invention relates to methods and pharmaceutical composition for the treatment of mucosal inflammatory diseases. In particular, the present invention is defined by the claims.

### DETAILED DESCRIPTION OF THE INVENTION

The mucosa is an integrated network of tissues, cells and effector molecules that protect the host from environmental insults and infections. Dysregulation of immunity at mucosal surfaces is thought to lead to mucosal inflammatory diseases such as those affecting the gastrointestinal system (Crohn's disease, ulcerative colitis and irritable bowel syndrome) and respiratory system (asthma, allergy and chronic obstructive pulmonary disorder). Anterior Gradient 2 (AGR2) is a dimeric Protein Disulfide Isomerase (PDI) family member involved in the regulation of protein quality control in the Endoplasmic Reticulum (ER). Its deletion in the mouse intestine increases tissue inflammation and promotes the development of inflammatory bowel disease (IBD). Now the inventors demonstrate that modulation of AGR2 dimer formation yields pro-inflammatory phenotypes notably though the secretion of AGR2 (eAGR2) that promotes monocyte attraction. The inventors show that in IBD and specifically in Crohn's disease, the levels of AGR2 dimerization modulators are selectively deregulated, and this correlates with severity of disease. The inventors thus demonstrate that AGR2 represent systemic alarm signals for pro-inflammatory responses in mucosa.

Accordingly, the present invention relates to a method of treating a mucosal inflammatory disease in a subject in need thereof comprising administering to the subject a therapeutically effective amount of an agent which neutralizes the pro-inflammatory activity of eAGR2.

As used herein, the term “mucosal inflammatory disease” has its general meaning in the art and refers to any disease characterised by a “mucosal inflammation”, which refers to swelling or irritation of the mucosa. As used, the term “mucosa” has its general meaning in the art and denotes the moist tissue lining body cavities which secretes mucous and covered with epithelium. Examples of mucosa tissue include, but are not limited to, oral mucosa e.g. buccal and sublingual; nasal mucosa; eye mucosa; genital mucosa; rectal mucosa; aural mucosa; lung mucosa; bronchial mucosa; gastric mucosa; intestinal mucosa; olfactory mucosa; uterine mucosa; and esophageal mucosa.

In some embodiments, the mucosal inflammatory disease affects the gastrointestinal system and typically includes inflammatory bowel diseases (IBD) such as Crohn's disease, ulcerative colitis and irritable bowel syndrome. The term “inflammatory bowel disease” or “IBD” is used as a collective term for ulcerative colitis and Crohn's disease. The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions, and the development of fistulas and fissures are not uncommon. As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine). The term “ulcerative colitis” or “UC” is used herein to refer to a condition involving inflammation of the large intestine and rectum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon. In some embodiments, the method of the present invention is particularly suitable for the treatment of colonic Crohn's disease. As used herein, the term “colonic Crohn's disease”, alternatively referred to as colonic CD, as used herein, means Crohn's disease where the inflammation is substantially localized to the colon.

In some embodiments, the mucosal inflammatory disease affects the respiratory system and typically includes asthma and chronic obstructive pulmonary disorder. As used herein, the term “asthma” refers to diseases that present as reversible airflow obstruction and/or bronchial hyper-responsiveness that may or may not be associated with underlying inflammation. Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas as mentioned, e.g., in the Expert Panel Report 3: Guidelines for the Diagnosis and Management of Asthma, National Asthma Education and Prevention Program (2007) (“NAEPP Guidelines”), incorporated herein by reference in its entirety. As used herein, the term “COPD” as used herein refers to chronic obstructive pulmonary disease. The term “COPD” includes two main conditions: emphysema and chronic obstructive bronchitis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “AGR2” has its general meaning in the art and refers to the gene encoding for the anterior gradient 2, protein disulphide isomerase family member (Gene ID:10551). The genomic sequence is referenced in the NCBI database under the NC_000007.14 accession number. An exemplary amino acid sequence for the human AGR2 is represented by SEQ ID NO:1.

>sp|O95994|AGR2_HUMAN Anterior gradient protein 2
homolog OS = Homo sapiens OX = 9606 GN = AGR2
PE = 1 SV = 1
SEQ ID NO: 1
MEKIPVSAFLLLVALSYTLARDTTVKPGAKKDTKDSRPKLPQTLSRGWG
DQLIWTQTYEEALYKSKTSNKPLMIIHHLDECPHSQALKKVFAENKEIQ
KLAEQFVLLNLVYETTDKHLSPDGQYVPRIMFVDPSLTVRADITGRYSN
RLYAYEPADTALLLDNMKKALKLLKTEL

As used herein, the term “eAGR2” refers to the secreted form of AGR2 such as described in Fessart, D., et al. Secretion of protein disulphide isomerase AGR2 confers tumorigenic properties. Elife 5(2016). eAGR2 deems to have the same amino acid sequence as described for AGR2.

In some embodiments, the expression “agent which neutralizes the pro-inflammatory activity of eAGR2” refers to any molecule that inhibits the recruitment of monocytes induced by eAGR2. The agent may be a small organic molecule or any biological molecule. Assays for determining whether a molecule can neutralize the pro-inflammatory activity of eAGR2 may be performed as those disclosed in the EXAMPLE section of the present specification. In some embodiments, the agent is an antibody specific for eAGR2.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “epitope” refers to a specific arrangement of amino acids located on a protein or proteins to which an antibody binds. Epitopes often consist of a chemically active surface grouping of molecules such as amino acids or sugar side chains, and have specific three-dimensional structural characteristics as well as specific charge characteristics. Epitopes can be linear or conformational, i.e., involving two or more sequences of amino acids in various regions of the antigen that may not necessarily be contiguous.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention an antibody fragment. As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, single chain antibody molecules, in particular scFv antibody fragments, disulfide-linked Fvs (sdFv), a Fd fragment consisting of the VH and CHI domains, linear antibodies, single domain antibodies such as, for example, sdAb (either VL or VH), camelid VHH domains, multi-specific antibodies formed from antibody fragments such as, for example, a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (see U.S. Pat. No. 6,703,199, which describes fibronectin polypeptide minibodies). Papain digestion of antibodies produces two identical antigen-binding fragments, called “Fab” fragments, and a residual “Fc” fragment, a designation reflecting the ability to crystallize readily.

Fragments and derivatives of antibodies of this invention (which are encompassed by the term “antibody” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

For instance, Fab or F(ab′)₂ fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 5 73 (2): 175-182 (1996), the disclosures of which are incorporated herein by reference.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”.

In some embodiments, the antibody comprises human heavy chain constant regions sequences but will not induce antibody dependent cellular cytotoxicity (ADCC). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

In some embodiments, the antibody of the present invention is 18A4 or one of its derivative form including the humanized form of said antibody as described in the following references, the contents of which are incorporated herein by reference:
Guo, Hao, et al. “A humanized monoclonal antibody targeting secreted anterior gradient 2 effectively inhibits the xenograft tumor growth.” Biochemical and biophysical research communications 475.1 (2016): 57-63.
Guo, H., et al. “Tumor-secreted anterior gradient-2 binds to VEGF and FGF2 and enhances their activities by promoting their homodimerization.” Oncogene 36.36 (2017): 5098.
Qudsia, Sehar, et al. “A novel lentiviral scFv display library for rapid optimization and selection of high affinity antibodies.” Biochemical and biophysical research communications 499.1 (2018): 71-77, and
US20140328829 Dawei Li, Zhenghua Wu, Hao GuoQi Zhu, Dhahiri S. Mashausi “Agr2 blocking antibody and use thereof”

In some embodiments, the antibody of the present invention is the murine anti-human monoclonal antibody 18A4 or humanized or chimeric form thereof. The 18A4 antibody is obtainable from the hybridoma cell line that was deposited in the China Center of Type Cell Collection (CCTCC) on Jan. 19, 2009 with a deposit number of CCTCC-C200902 at the address of the Wuhan University, Luojiashan, Wuchang, Wuhan, Hubei Province.

In some embodiments, the antibody of the present invention binds to an epitope that is located within the protein disulfide isomerase active domain of AGR2. In some embodiments, the antibody of the invention binds to an epitope comprising 1, 2, 3, 4, 5, 6, 7, 8, 9, 10, 11, 12, 13, 14, 15, 16, 17, 18, 19, 20, 21, or 22 in the amino acid sequence as set forth in SEQ ID NO:2 (PLMIIHHLDECPHSQALKKVFA). In some embodiments, the antibody of the present invention binds to an epitope as set forth in SEQ ID NO:2.

In some embodiments, the antibody of the invention comprises a heavy chain comprising at least one or at least two of the following CDRs:

H-CDR1:
(SEQ ID NO: 3)
DYNMD
H-CDR2:
(SEQ ID NO: 4)
DINPNYDTTSYNQKFQG
H-CDR3:
(SEQ ID NO: 5)
SMMGYGSPMDY

In some embodiments, the antibody of the invention comprises a light chain comprising at least one or at least two of the following CDRs:

L-CDR1:
(SEQ ID NO: 6)
RASKSVSTSGYSYMH
L-CDR2:
(SEQ ID NO: 7)
LASNLES
L-CDR3:
(SEQ ID NO: 8)
QHIRELPRT

In some embodiment, the antibody of the invention comprises a heavy chain comprising at least one of the following CDR i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and/or a light chain comprising at least one of the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

In some embodiment, the antibody of the invention comprises a heavy chain comprising the following CDR: i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and a light chain comprising the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

In some embodiments, the antibody of the present invention comprises the heavy chain as set forth in SEQ ID

QVQLVQSGAEVKKPGASVKVSCKASGYTFTDYNMDWVRQAPGQGLEWIG
DINPNYDTTSYNQKFKGKATLTVDKSTSTAYMELSSLRSEDTAVYYCAR
SMMGYGSPMDYWGQGTLVTVSS

In some embodiments, the antibody of the present invention comprises a heavy chain as set forth in SEQ ID NO:9 mutated by four substitutions at positions 65, 67, 68 and 70, wherein said substitutions are characterized in that:
lysine (K) at position 65 is changed to glutamine (Q),
lysine (K) at position 67 is changed to arginine (R),
alanine (A) at position 68 is changed to valine (V), and
leucine (L) at position 70 is changed to methionine (M), and

wherein the numbers of the positions correspond to the Kabat numbering system.

In some embodiments, the antibody of the present invention comprises the light chain as set forth in SEQ ID NO: 10:

EIVLTQSPATLSLSPGERATLSCRASKSVSTSGYSYMHWYQQKPGQAPR
LLIYLASNLESGIPARFSGSGSGTDFTLTISRLEPEDFAVYYCQHIREL
PRTFGGGTKLEIK

In some embodiments, the antibody of the present invention is selected among the antibodies described in Arumugam, Thiruvengadam, et al. “New Blocking Antibodies against Novel AGR2-C4. 4A Pathway Reduce Growth and Metastasis of Pancreatic Tumors and Increase Survival in Mice.” Molecular cancer therapeutics 14.4 (2015): 941-951, the content of which is incorporated herein by reference.

In some embodiments, the antibody of the invention binds to an epitope comprising 1, 2, 3, 4, 5, 6, 7, 8, 9, 10, 11, 12, 13, 14, 15, 16, 17, 18, 19, 20, 21, 22, 23, 24, 25, 26, 27, or 28 in the amino acid sequence as set forth in SEQ ID NO:11 (IHHLDECPHSQALKKVFAENKEIQKLAEQ). In some embodiments, the antibody of the present invention binds to an epitope as set forth in SEQ ID NO:11.

In some embodiments, the antibody of the invention comprises a heavy chain comprising at least one or at least two of the following CDRs:

H-CDR1:
(SEQ ID NO: 12)
NYGMN
H-CDR2:
(SEQ ID NO: 13)
WINTDTGKPTYTEEFKG
H-CDR3:
(SEQ ID NO: 14)
VTADSMDY

In some embodiments, the antibody of the invention comprises a light chain comprising at least one or at least two of the following CDRs:

L-CDR1:
(SEQ ID NO: 15)
RSSQSLVHSNGN
L-CDR2:
(SEQ ID NO: 16)
IYLH
L-CDR3:
(SEQ ID NO: 17)
SQSTHVPLT

In some embodiment, the antibody of the invention comprises a heavy chain comprising at least one of the following CDR i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and/or a light chain comprising at least one of the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

In some embodiment, the antibody of the invention comprises a heavy chain comprising the following CDR: i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and a light chain comprising the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

In some embodiments, the antibody of the present invention cross-competes for binding to AGR2 with the antibody comprising a heavy chain comprising the following CDR: i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and a light chain comprising the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

In some embodiments, the antibody of the present invention cross-competes for binding to AGR2 with the antibody comprising a heavy chain comprising the following CDR: i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and a light chain comprising the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

As used herein, the term “cross-competes” refers to monoclonal antibodies which share the ability to bind to a specific region of an antigen. In the present disclosure the monoclonal antibody that “cross-competes” has the ability to interfere with the binding of another monoclonal antibody for the antigen in a standard competitive binding assay. Such a monoclonal antibody may, according to non-limiting theory, bind to the same or a related or nearby (e.g., a structurally similar or spatially proximal) epitope as the antibody with which it competes. Cross-competition is present if antibody A reduces binding of antibody B at least by 60%, specifically at least by 70% and more specifically at least by 80% and vice versa in comparison to the positive control which lacks one of said antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

According to the present invention, the cross-competing antibody as above described retain the activity of antibody comprising a heavy chain comprising the following CDR: i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and a light chain comprising the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

According to the present invention, the cross-competing antibody as above described retain the activity of antibody comprising a heavy chain comprising the following CDR: i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and a light chain comprising the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

Any assay well known in the art would be suitable for identifying whether the cross-competing antibody retains the desired activity. For instance, the assay described in EXAMPLE that consist in determining the ability of impeding monocytes migration would be suitable for determining whether the antibody retains said ability.

By a “therapeutically effective amount” is meant a sufficient amount of the agent of the present invention for the treatment of the mucosal inflammatory disease at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the agent of the present invention may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The agent of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: eAGR2-mediated monocytes attraction. A) Freshly isolated PBMCs were placed in Boyden chambers towards media conditioned by cells overexpressing AGR2 WT, E60A, Δ45 or AGR2 AA mutants and incubated for 24 h. Migrating cells were then characterized and quantified by flow cytometry using FCS/SSC parameters or CD14 monocyte marker. (*): p<0.05. B and C) Freshly isolated PBMC were placed in Boyden chambers towards media conditioned by cells overexpressing TMED2 and either AGR2 WT (B) or AGR2 AA mutant (C) and incubated for 24 h. Migrating cells were then characterized and quantified as indicated in FIG. 1A. (*): p<0.05. D) Freshly isolated PBMC were placed in Boyden chambers towards media conditioned by cells silenced for TMED2 and incubated for 24 h. Migrating cells were then characterized and quantified as indicated in FIG. 1A. (*): p<0.05. E) Freshly isolated PBMC were placed in Boyden chambers towards decreased doses of recombinant AGR2 and incubated for 24 h. CCL2 cytokine was used as positive control for monocyte migration. ns: non-statistically significant, (*): p<0.05, (**): p<0.01, (***): p<0.005. F) Impact of AGR2 blocking antibodies on AGR2-mediated monocytes migration was tested using Boyden chambers as described above. The concentrations of recombinant human AGR2 was of 200 ng/ml and decreasing amounts of antibodies were used from 20 μg to 1 μg. The non-relevant antibody (Isotype) was used at the maximal dose of 20 μg. Data are representative of three independent experiments.

FIG. 2. Monocyte chemoattraction assays were performed using Boyden chambers. The impact of 3 anti-AGR2 antibodies (Clone 1C3, Abnova (10 ug); sc54569, Santacruz biotech (10 ug); home-made antibody (Pr Ted Hupp, CRUK) Mab3.4 (increasing doses)) was tested on AGR2-mediated monocyte chemoattraction. Naïve migration is presented in the white box, CCL2 mediated chemoattraction is used as a positive control. AGR2-mediated chemoattraction is shown. An isotype antibody (abnova) is used as negative control (ISO).

### EXAMPLE

Methods:

Materials—Tunicamycin (used at 2 μg/ml or otherwise indicated) was from Calbiochem (Guyancourt, France), thapsigargin (used at 500 nM or otherwise indicated) was from Calbiochem, Azetidine-2-carboxylic acid (used at 5 mM or otherwise indicated) and DTT (used at 0.5 mM or otherwise indicated) were from Sigma (St. Louis, Mo., USA). The siRNA library was from RNAi (http://mai.co.jp/lsci/products.html). DSP was from Thermo-Fisher—Pierce (Villebon-sur-Yvette, France).

Plasmid constructs—Constructs used in this report derived from the pcDNA5/FRT/TO (Invitrogen) plasmid. The segment encoding the transmembrane and cytosolic domains of IRE1 was cloned in pcDNA5/FRT/TO plasmid by standard PCR and restriction based cloning procedures. Baits and preys present in the hORFeome v8.1 were directly transferred in the pcDNA5/FRT/TO/IRE1 using the Gateway™ cloning technology (Life Technologies). The mutant constructions were obtained by PCR mutagenesis with the QuickChange® II Site-Directed Mutagenesis Kit (Agilent Technologies). The XBP1 splicing reporter was described previously (Samali et al., 2010). The hTMED2 expression plasmid was obtained from Sino Biological (HG13834-CF). The GFP-LC3 plasmid was a kind gift from Dr P. Codogno (Paris, France). AGR2 cDNA (WT, E60A, Δ45 and AA) were obtained from Genewiz (Sigma-Aldrich) and were cloned in pcDNA3.1 plasmids.

SiRNA screening—The screen was performed using a custom-made siRNA library targeting 274 ER resident proteins. Five thousand HEK293T cells were seeded in black 96-well plates. One day later, the cells were transfected with 200 μg of the AGR2/WT bait, 1.5 pmol of siRNA and 3 ng of the XBP1 luciferase reporter using the calcium phosphate precipitation procedure. In parallel, a counterscreen was performed by transfecting the siRNAs and the XBP1 luciferase reporter in the absence of the AGR2 WT bait. Two days after transfection, the luciferase activity was measured by chemiluminescence in an EnVision Multilabel Plate Reader (PerkinElmer, Waltham, Mass., USA). The raw values were log 2 transformed and were normalized to the average signal of the plate. The average negative signal of the plate was subtracted, separately for each replicate and a quantile normalization was performed. T-test and Kruskal-Wallis statistical analyses were performed to select the list of significant candidates.

Patients and sample analyses—Human ascending colon and ileal biopsies were obtained from the IBD Gastroenterology Unit, Beaujon Hospital. The protocol was in agreement with the local Ethics Committee (CPP-Ile de France IV No. 2009/17) and written informed consent was obtained from all the patients before inclusion. Thirty-two healthy controls, 8 patients with UC, and 40 patients with CD were selected (consecutively between 2012-2015) and included in this study. All patients were diagnosed based on classical clinical features as well as radiological, endoscopic, and histological findings. All biopsies were taken from the non-inflamed area of the right colon or the terminal ileum and analyzed by an expert GI pathologist. Unaffected areas were defined as mucosal regions without any macroscopic/endoscopic and histological signs of inflammation. To preserve tissue transcriptional profiles, biopsy specimens were kept at −80° C. until RNA extraction.

Immunohistochemistry. Paraffin-embedded sections of colon were deparaffinized in xylene, rehydrated, incubated in 3% hydrogen peroxide for endogenous peroxidase removal, and heated for 10 minutes in sub-boiling 10 mM citrate buffer (pH 6.0) for antigen retrieval. Then, sections were processed using the ImmPRESS reagent kit (Vector Laboratories). Primary antibodies against CD163 (AbCam, ab87099), TMED2 (Santa Cruz Biotechnology, sc-376459) and AGR2 (Novus Biologicals, NBP1-05936) were used.

Results:

AGR2 Forms Stress-Regulated Homodimer in the Endoplasmic Reticulum

Structural studies showed that AGR2 forms dimers through residues E60 (data not shown) and C81 (Patel et al., 2013; Ryu et al., 2012), respectively. Results involving E60 in AGR2 dimerization were confirmed using molecular dynamics (data not shown). The dimeric vs. monomeric equilibrium of AGR2 was also investigated using molecular modeling approaches. Indeed, the reduced dimer stability of the E60A mutant was verified by performing 200 ns molecular dynamics simulations of wild type and mutant dimers (data not shown). E60 of each monomer stabilizes the dimer by forming salt bridges to K64 of the other monomer. The WT system remains stable throughout the simulation, whereas the E60A mutant form rapidly dissociates, as identified in increased RMSD, Radius of Gyration and distance measurements, concomitant with loss of interaction energy. These results indicate that AGR2 might exist under both monomeric and homodimeric forms.

To validate the dimerization of AGR2 in our cellular models, cells were transfected with a previously validated siRNA against AGR2 (Higa et al., 2011) and its corresponding control siRNA. Cells were then treated with the chemical cross-linker DSP. Cross-linked proteins were resolved on either non-reducing (data not shown) or reducing (data not shown) conditions and analyzed by Western blot. Data revealed that AGR2 exists predominantly as homodimers. Since AGR2 is also involved in protein quality control in the ER (Higa et al., 2011), we evaluated the impact of ER homeostasis disruption on AGR2 dimerization. DSP-mediated protein crosslinking of tunicamycin-treated cells revealed that AGR2 homodimers disappeared upon ER stress induced by tunicamycin, whereas total AGR2 expression levels did not change significantly (data not shown).

To further dissect the mechanisms by which AGR2 dimerizes, we developed the ERMIT assay (data not shown). ERMIT is a mammalian two-hybrid method, adapted from the existing ER-MYTH yeast assay (Jansen et al., 2012) and based on the functional complementation of the IRE1 signaling pathway. IRE1 is normally maintained in an inactive state by its association with the molecular chaperone BiP. Upon accumulation of misfolded proteins in the ER, initiating ER stress, IRE1 competes with those proteins for binding to BiP. When activated, IRE1 cleaves XBP1 mRNA at two consensus sites to initiate an unconventional splicing reaction. This spliced mRNA leads to the generation of a functional XBP1 transcription factor (Hetz et al., 2015). In the ERMIT assay, the luminal domain of IRE1 was replaced by different bait proteins (data not shown) and independently of ER stress, bait and prey interactions leads to IRE1 activation and subsequent XBP1 splicing. This splicing is monitored by a XBP1 splicing luciferase reporter system (Hetz et al., 2015).

To determine if AGR2 dimerizes in the ER, we replaced the luminal domain of IRE1 with AGR2 wild-type (WT), or two AGR2 dimerization inactive mutants (E60A, C81A, or the E60A/C81A double mutant (DM)). The transmembrane and WT or kinase dead (KD) cytosolic domains of IRE1 were used as positive controls. These AGR2-IRE1 chimeric constructs were transfected into HEK293T cells and their expression and localization to the ER were verified by Western blot (data not shown) and immunofluorescence microscopy data not shown). ERMIT signals produced by HEK293T cells transfected with the different AGR2 baits were then quantified (data not shown). As IRE1 overexpression induces its auto-activation (Hetz et al., 2015), the ERMIT assay was optimized using low quantities of the transfected plasmids to ensure that no IRE1 auto-activation was detectable. In confirmation of the validity of the activation assay, all the IRE1 KD baits reduced the luminescence signal by more than 90% (data not shown), thus confirming that the signal observed was not due to the activation of endogenous IREL. The AGR2-WT bait produced the highest signal indicating that the dimerization of AGR2 occurred in the ER. The C81A mutant showed a 25% decrease in the signal, relative to AGR2-WT, whereas the E60A or the DM reduced the signal by about 80%. This demonstrates that AGR2 dimerizes in the ER and that the E60 residue plays a key role in this in vivo interaction whereas the C81 does not. Moreover, ER stress induced by DTT treatment showed a dose-dependent dissociation of AGR2 homodimers as assessed by the decrease in luminescence observed for all the constructs tested (data not shown). The same result was observed when ER stress was induced by thapsigargin or tunicamycin (data not shown). An IC50 was then calculated for each of the ER stressors (data not shown).

Stress-related AGR2 functions in the ER were also evaluated using ³⁵S-methionine pulse-chase followed by AGR2 immunoprecipitation to investigate the dynamics of AGR2 binding to other partners. Five AGR2 binding partners were visualized using this method in HeLa cells (data not shown). Interestingly, the kinetics of association of these proteins with AGR2 differed between basal and ER stress conditions. The association of the proteins corresponding to bands 2, 3 and 4 with AGR2 was destabilized upon ER stress, while that of proteins corresponding to bands 1 and 5 was stabilized (data not shown). These data led us to propose a model in which AGR2 exists mainly as a homodimer when protein-folding demand does not overwhelm the cellular folding capacity but in case of stress, AGR2 homodimers dissociate to unveil their chaperone/quality control properties. Moreover, our data also suggest that the ratio of monomeric versus dimeric AGR2 might represent a potent mean to selectively control ER proteostasis.

Identification of AGR2 Dimer Regulators and Functional Characterization of TMED2

To characterize the mechanisms regulating AGR2 dimeric vs. monomeric status, we designed a specific ERMIT-based siRNA screen and tested the impact of a custom-designed siRNA library that targets 274 ER resident proteins (data not shown). The counter-screen used cells transfected only with the XBP1s reporter (data not shown). We identified siRNAs that are positively or negatively modulating AGR2 dimer formation and allowed the identification of proteins that act as either inhibitors or enhancers of dimerization. A total of 71 proteins representing candidate AGR2 homodimer enhancers (42) or inhibitors (29) were identified (data not shown). Functional pathway analysis based on Gene Ontology and Reactome annotations of these candidates revealed an enrichment of AGR2 homodimer enhancers in protein productive folding and ERAD processes, while AGR2 homodimer inhibitors were significantly enriched in functions related to calcium homeostasis, ER stress and cell death processes (data not shown). Remarkably, a high network connectivity was observed between dimer enhancers (data not shown) or inhibitors (data not shown), thereby confirming AGR2 functions in productive protein folding when dimeric and managing misfolded proteins (stress) when monomeric. These data also confirm our primary hypothesis and reinforce the importance of AGR2 dimerization control in proper functioning of the ER.

Among the positive regulators of AGR2 dimerization found in the screen (data not shown), we identified TMED2, a p24 family member previously shown to function as a cargo receptor (Barlowe, 1998). Moreover, p24 family members in the yeast S. cerevisiae were shown to interact with PDI, the family of proteins to which AGR2 belongs (data not shown). To further characterize the functional interaction between TMED2 and AGR2, we first evaluated whether these 2 proteins could be found in a complex. As such co-immunoprecipitations were carried out under basal and ER stress conditions, either from HEK293T control cells or cells treated with tunicamycin (data not shown), or from a mouse ligated colonic loop model before and after treatment with tunicamycin (data not shown). The mouse colon was chosen as both AGR2 and TMED2 are highly expressed in this tissue. Both in vitro and in vivo, AGR2 was found in a complex with TMED2 that dissociated upon ER stress (data not shown). This observation suggests that under basal and stress conditions AGR2 is present in different functional complexes, a result supported by our siRNA and proteomic screens (Higa et al., 2011), where AGR2 mainly contributed to import into the ER, export to the Golgi apparatus or to ERAD (data not shown). The possible interaction of AGR2 monomer and dimer with TMED2 was explored using extensive protein-protein docking (data not shown). The identified interaction orientations between TMED2 and AGR2 monomer are for the most part unstable, and will block the possibility of AGR2 dimer formation (data not shown). Docking between TMED2 and AGR2 dimer, on the other hand, rendered several conformers in which TMED2 simultaneously interacts with both AGR2 monomers in the N-terminal/dimer interface regions (data not shown), and where perfect complementarity between structures and electrostatic surfaces of the two are noted (data not shown). We next examined the mechanisms underlying TMED2 regulation of AGR2 homodimerization. TMED2 overexpression led to enhanced AGR2 homodimer formation as evaluated using DSP-mediated cross-linking (data not shown). To further characterize the functional role of the interaction between TMED2 and AGR2, we sought to generate a mutant AGR2 unable to interact with TMED2, thereby not directly affecting TMED2 functions. To this end a molecular modeling approach was undertaken to identify amino-acid residues involved in the TMED2-AGR2 interaction and revealed that K66 and Y111 might play key roles (data not shown). As such, K66 and Y111 were mutated to alanine residues (referred to as AGR2 AA hereafter) and the interaction between AGR2 and TMED2 was evaluated using co-immunoprecipitation. As expected, whereas AGR2w and TMED2 co-immunoprecipitated, the interaction between TMED2 and AGR2 AA was impaired (data not shown). We next monitored the impact of TMED2 expression alteration on AGR2 level. Interestingly, overexpression of TMED2 led to reduced expression of AGR2 (data not shown), and reduced ERMIT signals, correlative to the loss of expression (data not shown). In contrast, the silencing of TMED2 led to enhanced expression of AGR2 (data not shown), but decreased ERMIT signals, indicative of effective dimerization inhibition (data not shown).

AGR2 Dimerization Ability does not Affect its Chaperone Functions but Alters its Localization

To explore the functional relevance of AGR2 dimerization, we tested how AGR2 regulates cargo secretion. As such the previously described interactions of AGR2 with the two plasma-membrane GPI-anchored proteins CD59 and LYPD3, that were reported in proteomics studies, were confirmed using ERMIT with the monomeric AGR2 E60A used as bait and either CD59 or LYPD3 used as preys, OS9 was used as a negative control (data not shown). Furthermore, we monitored the AGR2 contribution to the ER quality control and protein secretion using CD59 WT and mutant form, CD59 C94S. The latter due to its misfolding is no longer efficiently exported to the cell membrane and accumulates in the ER lumen (data not shown) even though the expression levels are similar (data not shown). We also found that both AGR2 WT and AA interacted with GFP-CD59 WT or C94S (data not shown). Interestingly, the modification of AGR2 and TMED2 expression levels impacted on CD59 degradation and trafficking (data not shown). Indeed, although AGR2 silencing led to reduced expression of intracellular CD59 WT (25%) and CD59 C94S (50%), it did not impact further on the expression of both proteins at the cell surface, thereby suggesting a role of intracellular AGR2 in quality control in the ER (data not shown). TMED2 silencing led to reduced expression of intracellular CD59 (either WT or C94S) and a similar effect was observed for cell surface expression (data not shown). These data indicated that the interplay between AGR2 and TMED2 exerts a selective regulation on protein folding and trafficking and contributes to protein quality control in the ER. To test the functionality of AGR2 AA, rescue experiments were carried out and showed that overexpression of either AGR2 WT or AGR2 AA restored the expression of GFP-CD59 (WT or C94S) total and at the cell surface (data not shown), thereby indicating that AGR2 AA conserved its ability to participate to ER folding and quality control mechanisms.

Further, we sought to investigate the impact of AGR2 on the secretion of cargo proteins under normal and ER stress conditions. Given that AGR2 peptide binding sites are present on alpha-1-antitrypsin (A1AT) (data not shown), we tested if AGR2 impacts on the secretion of this cargo. We examined the effect of silencing of AGR2 on secretion of A1AT by immunoblot under basal and stress conditions (data not shown). Under basal conditions, AGR2 was not involved in the secretion of A1AT as the silencing of AGR2 did not affect the kinetics of A1AT secretion. However, upon ER stress the retention of A1AT in the ER was decreased in the absence of AGR2. This suggests that AGR2 might also be involved in sensing ER homeostasis. Lastly, the presence of AGR2 stabilized the expression of MUC2 in HT29 cells, further confirming a crucial role for AGR2 in ER proteostasis. In addition, treatment of HT29 cells with the PTTIYY peptide (AGR2 binding; (Clarke et al., 2011)) rescued MUC2 expression upon ER stress (data not shown), suggesting the importance of the AGR2/MUC2 interaction in MUC2 quality control.

Since we observed an impact of TMED2 expression changes on iAGR2 expression levels, we sought to investigate the underlying molecular mechanisms involved in this phenomenon. First, the effects of overexpression of TMED2, which seemed to decrease the levels of intracellular AGR2 (iAGR2; data not shown) were not reversed by ERAD pharmacological inhibitors (data not shown). However, we found that this occurred through an alternative degradation mechanism involving autophagy (data not shown) and was reversed by chloroquine treatments (data not shown). This pointed towards an lysosomal/autophagy-dependent degradation of AGR2 induced by TMED2 overexpression. However, when we tested the presence of AGR2 in the extracellular milieu, we detected an anti-AGR2 immunoreactive band with an unexpected electrophoretic mobility (˜37 kDa; data not shown). This indicated that cells overexpressing TMED2 might present aberrant secretion features. This was confirmed by analyzing the insoluble material released by TMED2 overexpressing cells using cryo-electron microscopy that presented a very heterogenous profile of extracellular vesicles (and CD63 staining) compared to control cells (data not shown). Collectively these data show that overexpression of TMED2 leads to the abnormal secretory features including the release of aberrant AGR2 entities. TMED2 silencing, on the other hand, resulted in the increase of the intracellular fraction of AGR2 (iAGR2, data not shown) and promoted elevated AGR2 secretion in the medium (eAGR2; data not shown). Finally, we tested how constitutively monomeric (E60A) or dimeric (Δ45) AGR2 form behaved regarding secretion. Our results indicate that AGR2 E60A was secreted more efficiently than AGR2 WT and in the contrary, AGR2 Δ45 was retained inside the cell (data not shown). Importantly, TMED2 overexpression or silencing did not impact further the secretion of AGR2 AA (data not shown) thereby demonstrating the dependency of AGR2/TMED2 interactions for AGR2 secretion. Together, these results indicate that alteration of AGR2 dimeric vs. monomeric status impacts on AGR2 release in the extracellular milieu (either as a part of an altered secretory material or as a monomer).

Pathophysiological Implication of AGR2 Dimerization Control

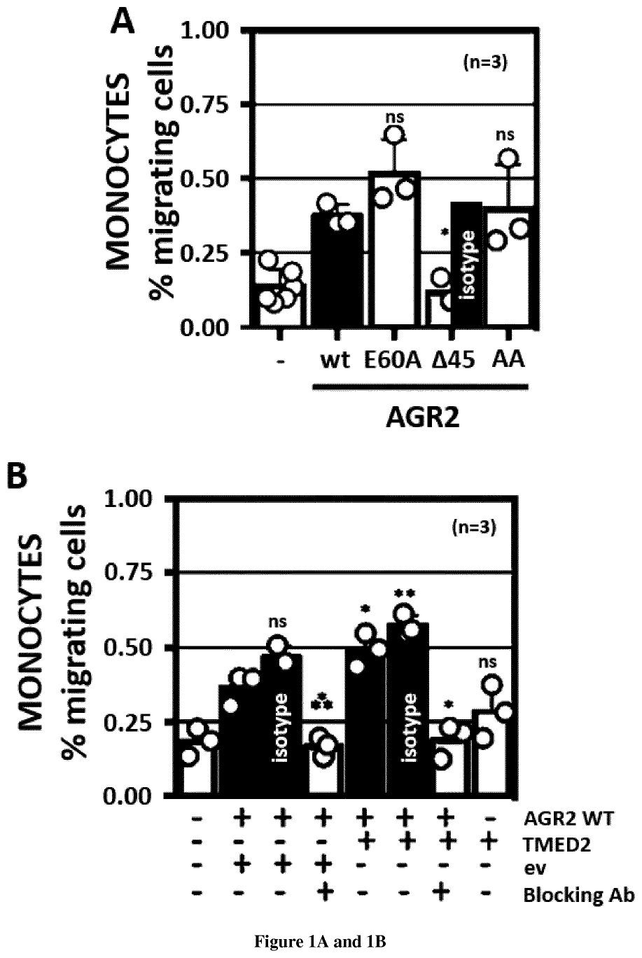

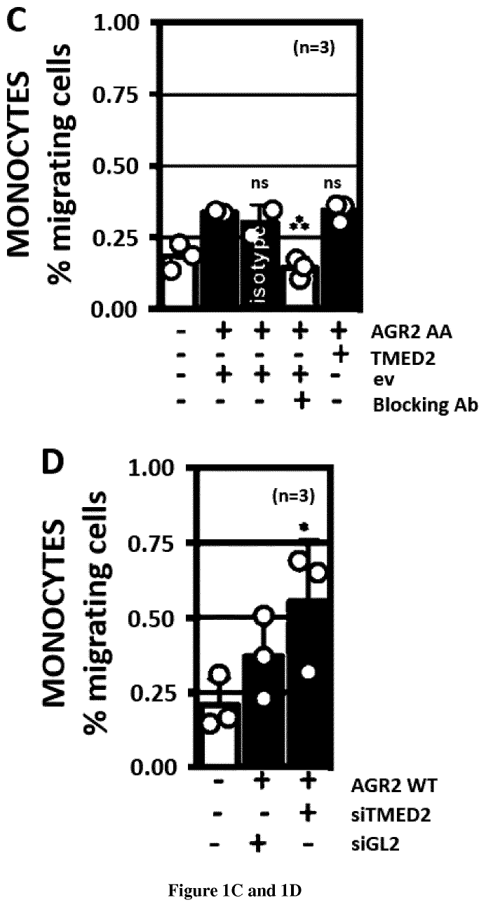

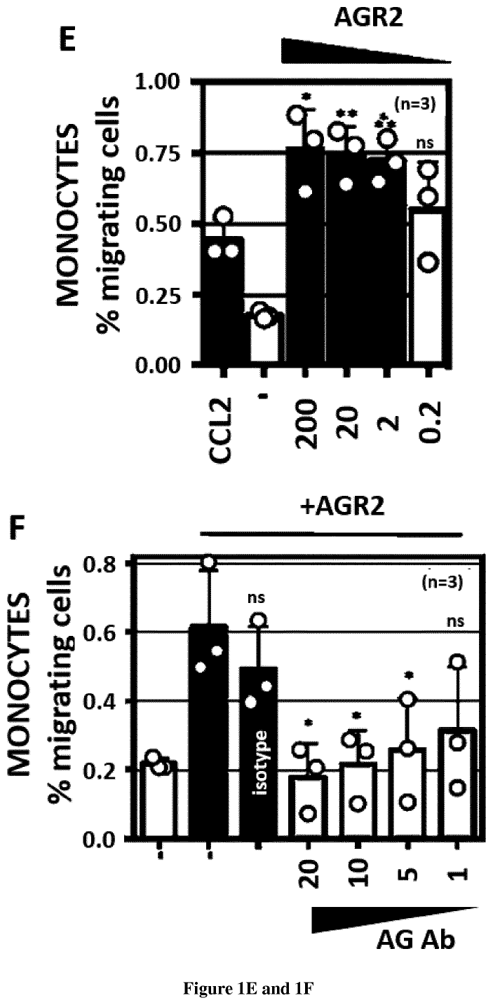

Since AGR2 was involved in hypersensitivity of intestinal epithelium to inflammation (Zhao et al., 2010) and since TMED2 was found to regulate AGR2 dimeric status, we postulated that mice exhibiting altered TMED2 expression should also display an intestinal phenotype. To test this hypothesis, we evaluated the expression of AGR2 and MUC2 in the intestine of mice expressing lower levels of TMED2 (heterozygous deficient; (Hou et al., 2017)). Interestingly typical signs of chronic intestinal inflammation were observed in TMED2 hypomorph mice such as loss of mucosecretion, inflammatory cell infiltrate, and hyperproliferation of mucosa in both the proximal colon and ileum (data not shown). Furthermore, TMED2 hypomorph mice exhibited lower global expression level of both AGR2 and MUC2 than WT mice (data not shown), thereby partly phenocopying the results observed in AGR2 deficient mice. As we recently showed that eAGR2 could exert signaling properties on cells by inducing EMT programs (Fessart et al., 2016), and since in our cellular models TMED2 silencing led to enhanced released of eAGR2, we reasoned that eAGR2 might also play a role in the chemoattraction of pro-inflammatory cells. To determine the direct involvement of eAGR2 in chemoattraction, PBMCs purified from three independent healthy donors were exposed either to media conditioned by cells overexpressing AGR2 WT, E60A, Δ45 or AA. Chemoattraction of monocytes from PBMCs was observed only when AGR2 was found in the extracellular milieu, namely when conditioned media from cells transfected with AGR2 WT, E60A or AA was used (FIG. 1A). Similar results were obtained when using media from cells overexpressing AGR2 WT or AA and simultaneously overexpressing TMED2 (FIGS. 1B and 1C), media from cells silenced for TMED2 (FIG. 1D) or even recombinant human AGR2 (FIG. 1E). Remarkably, AGR2 blocking antibodies were able in all cases to impede monocytes migration (FIGS. 1B, 1C, 1F). These experiments revealed that in all cases, eAGR2 was able to selectively promote monocyte attraction, thereby linking eAGR2 to pro-inflammatory phenotypes and unraveling the extracellular gain-of-function of AGR2 as a pro-inflammatory chemokine. Collectively, our results link the interaction between TMED2 and AGR2 and by extend the monomeric vs. dimeric status of AGR2 to pro-inflammatory phenotypes in the intestine. To test the relevance of these results in human IBD, we first evaluated the expression levels of the pathophysiological relevance of AGR2 dimer regulators in colonic biopsies from patients with IBD. Fifty-two of the 71 candidates as identified above were first tested in non-inflamed colonic biopsies from healthy controls, patients with ulcerative colitis (UC) and patients with Crohn's disease (CD), the two main classes of IBDs (data not shown). Messenger RNA expression levels of 12 out of 52 genes were found to be significantly different in CD while only 3 showed significant differences in UC (data not shown). The expression differences in AGR2 modulators were exacerbated in colonic CD patients (CC) (data not shown). To corroborate these findings, a validation cohort consisting of healthy controls and patients with ileo-colonic CD was used to evaluate mRNA expression levels of the 52 genes of interest. Fourteen genes, including the 12 genes previously identified, were significantly different in patients with CD, supporting the initial findings (data not shown). This allowed for the differentiation of CD patients from healthy controls (data not shown). Moreover, a functional enrichment analysis revealed that 6 genes whose silencing disrupted AGR2 dimer formation were either up-regulated or down-regulated in CD (namely TMED2, RPN1, KTN1, LMAN1, AMFR, AKAP6) and that 4 genes whose silencing promoted AGR2 dimerization were systematically down-regulated in CD (namely P4HTM, SYVN3, CES3, SCAP). TMED2 mRNA (data not shown) and protein (data not shown) expression was increased in CD, mainly in normal intestinal epithelial cells. TMED2 overexpression was detected in patients with active (A) CD and correlated with high recruitment of CD163 positive macrophages in the colonic mucosa (data not shown). Remarkably, patients with quiescent (Q) CD exhibited a moderate loss of AGR2 global staining which likely correlated with its probable secretion (data not shown). These data indicate that regulation of AGR2 dimerization is associated with pro-inflammatory responses and enrichment of macrophages in the colonic mucosa that could be observed in CD. Dissecting the diversity and the local distribution of functional macrophages in patients with active or quiescent CD will further define clinical relevance of AGR2.

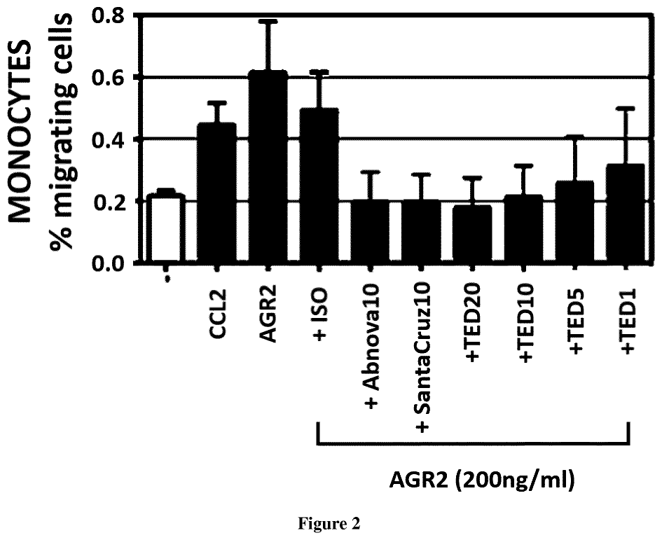

Moreover, the impact of 3 anti-AGR2 antibodies (Clone 1C3, Abnova (10 ug); sc54569, Santacruz biotech (10 ug); home-made antibody (Pr Ted Hupp, CRUK) Mab3.4 (increasing doses)) was tested on AGR2-mediated monocyte chemoattraction (FIG. 2). AGR2 blocking antibodies were able to impede monocytes migration.

Discussion:

The results presented in this study show that in the ER, AGR2 exists under monomeric or dimeric configurations and modulation of AGR2 dimeric vs. monomeric status might represent a novel ER proteostasis sensor mechanism in intestinal epithelial cells. Moreover, we identify a mechanism of regulation of AGR2 dimerization through an interaction with the protein TMED2. Furthermore, our data link the perturbation of AGR2 dimerization to inflammatory bowel disease in human in part through the unexpected intervention of AGR2 in the recruitment of inflammatory cells. Collectively, our results document a molecular link between ER proteostasis control and a pro-inflammatory systemic stress response which when abnormal turns out as a disease state in the colon.

We first reasoned that since an excess of AGR2 dimers or AGR2 monomers yields a pro-inflammatory response, a systemic adaptive reaction, the relative concentrations of each form might be linked to proper function of the early secretory pathway. In this context, dysregulation of the relative equilibrium of AGR2 dimers and monomers could be a sign of ER proteostasis imbalance. In the context of IBD, protein homeostasis within the early secretory pathway and its adaptation to the perturbation through the UPR, have been shown to play instrumental roles in disease onset (Grootjans et al., 2016). In the present work, we identified AGR2 as a critical player in such adaptive mechanism and we further demonstrated that under basal conditions AGR2 mainly interacts with Golgi export components to ensure proper protein folding, while during ER stress it forms functional complexes with ERAD machinery to clear the misfolded proteins from the ER. Moreover, this study provides the identification of AGR2 status, monomer vs. dimer balance, as an early event possibly able to define the extent and some characteristics of intestinal inflammation. This is particularly appealing for IBD, which is characterized by the chronic inflammation and ulceration of the gastrointestinal tract due to an overactive immune digestive system. Our data suggest that perturbation of AGR2 dimerization, due to variable expression levels of its client proteins, can lead to IBD development. This could actually be relevant at several levels through the release of extracellular AGR2 which might as previously found in other models induce Epithelial-to-Mesenchymal Transition markers¹³ to promote fibrosis which is a hallmark of Crohn's disease and in the mean-time to promote the recruitment of macrophages to the site of damage to precondition the tissue for uncontrolled inflammation.

Interestingly, our results also establish that the interaction between AGR2 and TMED2 plays a key role in AGR2 dimerization control by stabilizing the dimer. The alteration of TMED2 expression in mice, resulting from the heterozygous expression of a mutant form of the protein that is not properly synthesized (Hou et al., 2017) resulted in alteration of colon homeostasis and inflammation. Moreover, overexpression of TMED2 was detected in active CD and may also be associated with inflammation through autophagy-dependent AGR2 release in the extracellular milieu (Park et al., 2009; Zhao et al., 2010). A similar mechanism could be applied to other AGR2 expressing cells, such as pancreatic, biliary or lung epithelia. Findings from this study might be further applicable to cancer biology, since proteostasis imbalance has emerged as a major cancer hallmark, capable of driving tumor aggressiveness (Chevet et al., 2015). In light of our findings, control of AGR2 dimerization may well be a relevant factor in cancer development. High AGR2 expression, as well as its secretion into body fluids, was reported in many cancer types and associated with pro-tumorigenic phenotype and poor patient outcome (Brychtova et al., 2015; Chevet et al., 2013; Obacz et al., 2015). However, questions remain as to what is the predominant form of AGR2 in cancer cells, how is the formation of AGR2 dimer vs. monomer precisely regulated and what are the biological/functional consequences of AGR2 dimerization? These issues warrant deeper investigation. Collectively, our data provide the first evidence of the existence of ER sensors such as AGR2, that contribute to the regulation of proteostasis boundaries in this compartment, and whose alteration leads to pro-inflammatory responses.

### REFERENCES

Throughout this application, various references describe the state of the art to which this invention pertains. The disclosures of these references are hereby incorporated by reference into the present disclosure.
Barlowe, C. (1998). COPII and selective export from the endoplasmic reticulum. Biochim Biophys Acta 1404, 67-76.
Brychtova, V., Mohtar, A., Vojtesek, B., and Hupp, T. R. (2015). Mechanisms of anterior gradient-2 regulation and function in cancer. Semin Cancer Biol 33, 16-24.
Chang, C. L., Hong, E., Lao-Sirieix, P., and Fitzgerald, R. C. (2008a). A novel role for the retinoic acid-catabolizing enzyme CYP26A1 in Barrett's associated adenocarcinoma. Oncogene 27, 2951-2960.
Chang, J., Chance, M. R., Nicholas, C., Ahmed, N., Guilmeau, S., Flandez, M., Wang, D., Byun, D. S., Nasser, S., Albanese, J. M., et al. (2008b). Proteomic changes during intestinal cell maturation in vivo. J Proteomics 71, 530-546.
Chevet, E., Fessart, D., Delom, F., Mulot, A., Vojtesek, B., Hrstka, R., Murray, E., Gray, T., and Hupp, T. (2013). Emerging roles for the pro-oncogenic anterior gradient-2 in cancer development. Oncogene 32, 2499-2509.
Chevet, E., Hetz, C., and Samali, A. (2015). Endoplasmic reticulum stress-activated cell reprogramming in oncogenesis. Cancer Discov 5, 586-597.
Clarke, D. J., Murray, E., Hupp, T., Mackay, C. L., and Langridge-Smith, P. R. (2011). Mapping a noncovalent protein-peptide interface by top-down FTICR mass spectrometry using electron capture dissociation. J Am Soc Mass Spectrom 22, 1432-1440.
Fessart, D., Domblides, C., Avril, T., Eriksson, L. A., Begueret, H., Pineau, R., Malrieux, C., Dugot-Senant, N., Lucchesi, C., Chevet, E., et al. (2016). Secretion of protein disulphide isomerase AGR2 confers tumorigenic properties. Elife 5.
Grootjans, J., Kaser, A., Kaufman, R. J., and Blumberg, R. S. (2016). The unfolded protein response in immunity and inflammation. Nat Rev Immunol 16, 469-484.
Hetz, C., Chevet, E., and Oakes, S. A. (2015). Proteostasis control by the unfolded protein response. Nat Cell Biol 17, 829-838.
Higa, A., Mulot, A., Delom, F., Bouchecareilh, M., Nguyen, D. T., Boismenu, D., Wise, M. J., and Chevet, E. (2011). Role of pro-oncogenic protein disulfide isomerase (PDI) family member anterior gradient 2 (AGR2) in the control of endoplasmic reticulum homeostasis. J Biol Chem 286, 44855-44868.
Hou, W., Gupta, S., Beauchamp, M. C., Yuan, L., and Jerome-Majewska, L. A. (2017). Non-alcoholic fatty liver disease in mice with heterozygous mutation in TMED2. PLoS One 12, e0182995.
Jansen, G., Maattanen, P., Denisov, A. Y., Scarffe, L., Schade, B., Balghi, H., Dejgaard, K., Chen, L. Y., Muller, W. J., Gehring, K., et al. (2012). An interaction map of endoplasmic reticulum chaperones and foldases. Mol Cell Proteomics 11, 710-723.
Kaser, A., Adolph, T. E., and Blumberg, R. S. (2013). The unfolded protein response and gastrointestinal disease. Semin Immunopathol 35, 307-319.
Komiya, T., Tanigawa, Y., and Hirohashi, S. (1999). Cloning of the gene gob-4, which is expressed in intestinal goblet cells in mice. Biochim Biophys Acta 1444, 434-438.
Maslon, M. M., Hrstka, R., Vojtesek, B., and Hupp, T. R. (2010). A divergent substrate-binding loop within the pro-oncogenic protein anterior gradient-2 forms a docking site for Reptin. J Mol Biol 404, 418-438.
Obacz, J., Takacova, M., Brychtova, V., Dobes, P., Pastorekova, S., Vojtesek, B., and Hrstka, R. (2015). The role of AGR2 and AGR3 in cancer: similar but not identical. Eur J Cell Biol 94, 139-147.
Park, S. W., Zhen, G., Verhaeghe, C., Nakagami, Y., Nguyenvu, L. T., Barczak, A. J., Killeen, N., and Erle, D. J. (2009). The protein disulfide isomerase AGR2 is essential for production of intestinal mucus. Proc Natl Acad Sci USA 106, 6950-6955.
Patel, P., Clarke, C., Barraclough, D. L., Jowitt, T. A., Rudland, P. S., Barraclough, R., and Lian, L. Y. (2013). Metastasis-promoting anterior gradient 2 protein has a dimeric thioredoxin fold structure and a role in cell adhesion. J Mol Biol 425, 929-943.
Persson, S., Rosenquist, M., Knoblach, B., Khosravi-Far, R., Sommarin, M., and Michalak, M. (2005). Diversity of the protein disulfide isomerase family: identification of breast tumor induced Hag2 and Hag3 as novel members of the protein family. Mol Phylogenet Evol 36, 734-740.
Powers, E. T., Morimoto, R. I., Dillin, A., Kelly, J. W., and Balch, W. E. (2009). Biological and chemical approaches to diseases of proteostasis deficiency. Annu Rev Biochem 78, 959-991.
Ryu, J., Park, S. G., Lee, P. Y., Cho, S., Lee do, H., Kim, G. H., Kim, J. H., and Park, B. C. (2012). Dimerization of pro-oncogenic protein Anterior Gradient 2 is required for the interaction with BiP/GRP78. Biochem Biophys Res Commun 430, 610-615.
Samali, A., Fitzgerald, U., Deegan, S., and Gupta, S. (2010). Methods for monitoring endoplasmic reticulum stress and the unfolded protein response. Int J Cell Biol 2010, 830307.
Yu, H., Zhao, J., Lin, L., Zhang, Y., Zhong, F., Liu, Y., Yu, Y., Shen, H., Han, M., He, F., et al. (2013). Proteomic study explores AGR2 as pro-metastatic protein in HCC. Mol Biosyst 8, 2710-2718.
Zhao, F., Edwards, R., Dizon, D., Afrasiabi, K., Mastroianni, J. R., Geyfman, M., Ouellette, A. J., Andersen, B., and Lipkin, S. M. (2010). Disruption of Paneth and goblet cell homeostasis and increased endoplasmic reticulum stress in Agr2−/− mice. Dev Biol 338, 270-279.
Zheng, W., Rosenstiel, P., Huse, K., Sina, C., Valentonyte, R., Mah, N., Zeitlmann, L., Grosse, J., Ruf, N., Nurnberg, P., et al. (2006). Evaluation of AGR2 and AGR3 as candidate genes for inflammatory bowel disease. Genes Immun 7, 11-18.

1. A method of treating a mucosal inflammatory disease in a subject in need thereof comprising administering to the subject a therapeutically effective amount of an agent which neutralizes the pro-inflammatory activity of eAGR2.

2. The method of claim 1 wherein the subject suffers from an inflammatory bowel disease (IBD).

3. The method of claim 2 wherein the IBD is selected from the group consisting of Crohn's disease, ulcerative colitis and irritable bowel syndrome

4. The method of claim 1 wherein the subject suffers from a mucosal inflammatory disease that affects the respiratory system.

5. The method of claim 4 wherein the subject suffers from asthma or chronic obstructive pulmonary disorder.

6. The method of claim 1 wherein the agent is an antibody specific for eAGR2.

7. The method of claim 8 wherein the antibody binds to an epitope comprising 1, 2, 3, 4, 5, 6, 7, 8, 9, 10, 11, 12, 13, 14, 15, 16, 17, 18, 19, 20, 21, or 22 in the amino acid sequence as set forth in SEQ ID NO:2 (PLMIIHHLDECPHSQALKKVFA).

8. The method of claim 7 wherein the antibody binds to an epitope as set forth in SEQ ID NO:2.

9. The method of claim 7 wherein the antibody comprises a heavy chain comprising at least one or at least two of the following CDRs:
H-CDR1:
(SEQ ID NO: 3)
DYNMD
H-CDR2:
(SEQ ID NO: 4)
DINPNYDTTSYNQKFQG
H-CDR3:
(SEQ ID NO: 5)
SMMGYGSPMDY

10. The method of claim 7 wherein the antibody comprises a light chain comprising at least one or at least two of the following CDRs:
L-CDR1:
(SEQ ID NO: 6)
RASKSVSTSGYSYMH
L-CDR2:
(SEQ ID NO: 7)
LASNLES
L-CDR3:
(SEQ ID NO: 8)
QHIRELPRT

11. The method of claim 7 wherein the antibody comprises a heavy chain comprising at least one of the following CDR i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and/or a light chain comprising at least one of the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

12. The method of claim 7 wherein the antibody comprises a heavy chain comprising the following CDR: i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and a light chain comprising the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

13. The method of claim 7 wherein the antibody comprises the heavy chain as set forth in SEQ ID NO: 9.

14. The method of claim 7 wherein the antibody comprises a heavy chain as set forth in SEQ ID NO:9 mutated by four substitutions at positions 65, 67, 68 and 70, wherein said substitutions are characterized in that:
lysine (K) at position 65 is changed to glutamine (Q),
lysine (K) at position 67 is changed to arginine (R),
alanine (A) at position 68 is changed to valine (V), and
leucine (L) at position 70 is changed to methionine (M), and
wherein the numbers of the positions correspond to the Kabat numbering system.

15. The method of claim 7 wherein the antibody comprises the heavy chain as set forth in SEQ ID NO: 10.

16. The method of claim 8 wherein the antibody binds to an epitope comprising 1, 2, 3, 4, 5, 6, 7, 8, 9, 10, 11, 12, 13, 14, 15, 16, 17, 18, 19, 20, 21, 22, 23, 24, 25, 26, 27, or 28 in the amino acid sequence as set forth in SEQ ID NO:11
(IHHLDECPHSQALKKVFAENKEIQKLAEQ).

17. The method of claim 16 wherein the antibody binds to an epitope as set forth in SEQ ID NO:11.

18. The method of claim 16 wherein the antibody comprises a heavy chain comprising at least one or at least two of the following CDRs:
H-CDR1:
(SEQ ID NO: 12)
NYGMN
H-CDR2:
(SEQ ID NO: 13)
WINTDTGKPTYTEEFKG
H-CDR3:
(SEQ ID NO: 14)
VTADSMDY

19. The method of claim 16 wherein the antibody comprises a light chain comprising at least one or at least two of the following CDRs:
L-CDR1:
(SEQ ID NO: 15)
RSSQSLVHSNGN
L-CDR2:
(SEQ ID NO: 16)
IYLH
L-CDR3:
(SEQ ID NO: 17)
SQSTHVPLT

20. The method of claim 16 wherein the antibody comprises a heavy chain comprising at least one of the following CDR i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and/or a light chain comprising at least one of the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

21. The method of claim 16 wherein the antibody comprises a heavy chain comprising the following CDR: i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and a light chain comprising the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).

22. The method of claim 8 wherein the antibody cross-competes for binding to AGR2 with the antibody comprising a heavy chain comprising the following CDR: i) the VH-CDR1 as set forth in SEQ ID NO:3 (DYNMD), ii) the VH-CDR2 as set forth in SEQ ID NO:4 (DINPNYDTTSYNQKFQG) and iii) the VH-CDR3 as set forth in SEQ ID NO:5 (SMMGYGSPMDY) and a light chain comprising the following CDR: i) the VL-CDR1 as set forth in SEQ ID NO:6 (RASKSVSTSGYSYMH), ii) the VL-CDR2 as set forth in SEQ ID NO:7 (LASNLES) and iii) the VL-CDR3 as set forth in SEQ ID NO:8 (QHIRELPRT).

23. The method of claim 8 wherein the antibody cross-competes for binding to AGR2 with the antibody comprising a heavy chain comprising the following CDR: i) the VH-CDR1 as set forth in SEQ ID NO:12 (NYGMN), ii) the VH-CDR2 as set forth in SEQ ID NO:13 (WINTDTGKPTYTEEFKG) and iii) the VH-CDR3 as set forth in SEQ ID NO:14 (VTADSMDY) and a light chain comprising the following CDR: i) the VL-CDR1 as set forth in SEQ ID NO:15 (RSSQSLVHSNGN), ii) the VL-CDR2 as set forth in SEQ ID NO:16 (IYLH) and iii) the VL-CDR3 as set forth in SEQ ID NO:17 (SQSTHVPLT).


SEQUENCE LISTING (17 sequences)

SEQ ID NO: 1 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 2 - protein, 22 aa, Homo sapiens
PLMIIHHLDECPHSQALKKVFA

SEQ ID NO: 3 - protein, 5 aa, Artificial Sequence
  Synthetic H-CDR1
DYNMD

SEQ ID NO: 4 - protein, 17 aa, Artificial Sequence
  Synthetic H-CDR2
DINPNYDTTSYNQKFQG

SEQ ID NO: 5 - protein, 11 aa, Artificial Sequence
  Synthetic H-CDR3
SMMGYGSPMDY

SEQ ID NO: 6 - protein, 15 aa, Artificial Sequence
  Synthetic L-CDR1
RASKSVSTSGYSYMH

SEQ ID NO: 7 - protein, 7 aa, Artificial Sequence
  Synthetic L-CDR2
LASNLES

SEQ ID NO: 8 - protein, 9 aa, Artificial Sequence
  Synthetic L-CDR3
QHIRELPRT

SEQ ID NO: 9 - protein, 120 aa, Artificial Sequence
  Synthetic Heavy Chain Sequence
QVQLVQSGAEVKKPGASVKVSCKASGYTFTDYNMDWVRQAPGQGLEWIGDINPNYDTTSYNQKFKGKATLTVDKSTSTAYMELSSLRSEDTAVYYCARSMMGYGSPMDYWGQGTLVTVSS

SEQ ID NO: 10 - protein, 111 aa, Artificial Sequence
  Synthetic Light Chain Sequence
EIVLTQSPATLSLSPGERATLSCRASKSVSTSGYSYMHWYQQKPGQAPRLLIYLASNLESGIPARFSGSGSGTDFTLTISRLEPEDFAVYYCQHIRELPRTFGGGTKLEIK

SEQ ID NO: 11 - protein, 29 aa, Artificial Sequence
  Synthetic Epitope
IHHLDECPHSQALKKVFAENKEIQKLAEQ

SEQ ID NO: 12 - protein, 5 aa, Artificial Sequence
  Synthetic H-CDR1
NYGMN

SEQ ID NO: 13 - protein, 17 aa, Artificial Sequence
  Synthetic H-CDR2
WINTDTGKPTYTEEFKG

SEQ ID NO: 14 - protein, 8 aa, Artificial Sequence
  Synthetic H-CDR3
VTADSMDY

SEQ ID NO: 15 - protein, 12 aa, Artificial Sequence
  Synthetic L-CDR1
RSSQSLVHSNGN

SEQ ID NO: 16 - protein, 4 aa, Artificial Sequence
  Synthetic L-CDR2
IYLH

SEQ ID NO: 17 - protein, 9 aa, Artificial Sequence
  Synthetic L-CDR3
SQSTHVPLT